(19) United States
(12) Patent Application Publication
(10) Pub. No.: US 2017/0246419 A1
(43) Pub. Date: Aug. 31, 2017

(54) DEVICES, SYSTEMS, AND METHODS FOR MIXING AND BLENDING TWO OR MORE FLUIDS

(71) Applicant: ONEBREATH, INC., Palo Alto, CA (US)

(72) Inventors: Matthew John Callaghan, Palo Alto, CA (US); Lawrence Edward Miller, Palo Alto, CA (US); Edward Ayrapetian, Palo Alto, CA (US)

(73) Assignee: ONEBREATH, INC., Palo Alto, CA (US)

(21) Appl. No.: 15/516,502

(22) PCT Filed: Oct. 7, 2015
(86) PCT No.: PCT/US15/54518

§ 371 Date: Apr. 3, 2017

Related U.S. Application Data
(60) Provisional application No. 62/060,945, filed on Oct. 7, 2014.

(51) Int. Cl. A61M 16/12; A61M 16/00; A61M 16/10; A61M 16/20

(52) U.S. Cl. CPC A61M 16/12; A61M 16/203; A61M 16/208; A61M 16/0063; A61M 16/1005; A61M 2016/0027; A61M 2205/3368; A61M 2205/3331; A61M 2016/0039; A61M 2202/0208; A61M 2016/102; A61M 2205/502

(57) ABSTRACT

Embodiments disclosed herein are directed to devices, systems, and methods for mixing and/or blending two or more fluids, such as gases, to produce suitable mixed or blended fluids, such as a breathable gas. For example, the system may control and/or regulate flow from of first fluid from a first source and/or flow of a second fluid from a second source. The system may include a controller that may operate or direct operation of one or more valves to control the flow of the first and second fluids, thereby producing a blended or mixed fluid that has selected concentrations or proportions (or ratios) of the first and second fluids.

### CROSS-REFERENCE TO RELATED APPLICATIONS

This application claims priority to U.S. Provisional Application No. 62/060,945 filed on 7 Oct. 2014, the disclosure of which is incorporated herein, in its entirety, by this reference.

### BACKGROUND

Generally, a medical ventilator may be used to supply or force breathable gas (e.g., an air-oxygen mixture) into the lungs of a patient. The oxygen-air mixture is typically expressed in terms of oxygen concentration or fraction of inspired oxygen (FiO2). FiO2 is commonly expressed in increments, such as 1% to 21%, of atmospheric air to 100% oxygen.

The concentration of the mixture, or the FiO2 thereof, may be adjusted during the ventilation of the lungs. For example, as the condition of the patient improves, the concentration of oxygen may be reduced. Conversely, if the condition of the patient worsens, the patient may require a higher concentration of oxygen. Hence, providing breathable gas with a selected FiO2 may be important to treating the patient safely and effectively.

Accordingly, users and manufacturers of ventilation devices and systems continue to seek improvements to such ventilation devices.

### SUMMARY

Embodiments disclosed herein are directed to devices, systems, and methods for mixing and/or blending two or more fluids (e.g., as gases) to produce a suitable blended fluid, such as a breathable gas. In an embodiment, the system may include or couple to two or more fluid sources and may mix and/or blend the fluids therefrom. For example, the system may control and/or regulate flow from of first fluid from a first source and/or flow of a second fluid from a second source. The system may include a controller that may operate or direct operation of one or more valves to control the flow of the first and second fluids, thereby producing a blended or mixed fluid that has selected concentrations or proportions (or ratios) of the first and second fluids.

At least one embodiment includes a system for blending a first fluid and a second fluid to produce a blended fluid of a selected composition. The system includes an accumulator defining an accumulator volume, a first fluid supply branch, and a second fluid supply branch. The first fluid supply branch includes a first valve coupleable to a first source of pressurized fluid, and a first supply line coupled to the first input valve and extending downstream therefrom and being in fluid communication with the accumulator volume. The first fluid supply branch also includes a first mass flow sensor positioned along the first supply and configured to generate a first signal that corresponds to a mass of fluid at a location thereof. The second fluid supply includes a second valve coupleable to a second source of pressurized fluid and a second supply line coupled to the second input valve and extending downstream therefrom and being in fluid communication with the accumulator volume. The second supply branch also includes a second mass flow sensor positioned along the second supply line and configured to generate a second signal that corresponds to a mass of fluid at a location thereof. Moreover, the system includes a controller operably coupled to the first and second mass flow sensors and configured to receive signals therefrom. The controller is further configured to operate or direct operation of the first and the second valves, at least partially based on the received signals from the first and second mass flow sensors, to generate a flow of the first and second fluids targeted, to produce a blended fluid in the accumulator, which has selected concentrations of the first and second fluids.

Additional or alternative embodiments include a system for blending a first gas and a second gas to produce a breathable gas having a selected FiO2. The system includes an accumulator defining an accumulator volume, a first fluid supply branch, and a second fluid supply branch. The first fluid supply branch includes an electrically-controlled valve coupled to a source of the first gas and a first supply line coupled to the electrically-controlled valve and extending downstream therefrom and being in fluid communication with the accumulator volume. The second fluid supply includes a second supply line in fluid communication with the accumulator volume. In addition, the system includes a controller operably coupled to the first electrically-controlled valve and configured to direct progressive opening and closing thereof.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments, wherein identical reference numerals refer to identical or similar elements or features in different views or embodiments shown in the drawings.

FIG. 1 is a schematic diagram of a system for blending multiple fluids, according to an embodiment;

FIG. 2 is a schematic diagram of a system for blending multiple fluids, according to another embodiment;

FIG. 3 is a schematic diagram of a system for blending multiple fluids, according to one or more other embodiments;

FIG. 4 is a schematic diagram of a mass flow meter, according to an embodiment; and

FIG. 5 is a schematic diagram of a fluid flow control circuit, according to an embodiment.

### DETAILED DESCRIPTION

Embodiments disclosed herein are directed to devices, systems, and methods for mixing and/or blending two or more fluids (e.g., as gases) to produce a suitable blended fluid, such as a breathable gas. In an embodiment, the system may include or couple to two or more fluid sources and may mix and/or blend the fluids therefrom. For example, the system may control and/or regulate flow from of first fluid from a first source and/or flow of a second fluid from a second source. The system may include a controller that may operate or direct operation of one or more valves to control the flow of the first and second fluids, thereby producing a blended or mixed fluid that has selected concentrations or proportions (or ratios) of the first and second fluids.

In an embodiment, the system may include one or more sensors that may detect the amount or mass of fluid flowing from two or more sources of fluid to a mixing location. The controller may receive signals from the sensor(s) and may operate or direct operation of valves(s) that may at least partially open or close flow of the fluid(s) from selected source(s), such that selected amounts of the first and second fluids flow from the respective sources thereof to the mixing location. For example, the controller open the valve(s) to a predetermined opening to produce a suitable flow and/or periodically open and close valve(s) such as to permit a selected amount of fluid to flow therethrough and to the mixing location.

Generally, the system may blend together any number of fluids (e.g., liquids, gases, liquid-gas mixtures, etc.) to produce a blended fluid that may vary from one embodiment to the next. Moreover, the system may blend together multiple fluids to produce a blended fluid that has any number of suitable or selected properties. For example, the system may blend together two or more fluids to produce a blended fluid that has selected concentrations or ratio(s) of masses of the constituent fluids. Additionally or alternatively, two or more fluids may be mixed or blended together to produce a blended fluid that has a selected pH, color, volumetric ratios of the constituent fluids, combinations of the foregoing, etc.

In some embodiments, the mixing location may be an accumulator. For example, first and second fluids may flow from their respective sources thereof and into the accumulator to produce a blended fluid (e.g., a breathable gas having a selected Oxygen concentration or a ratio by mass, such as a selected FiO2). The accumulator may include an output outlet and/or an output valve, and the blended gas may be directed from the accumulator to a selected location (e.g., lungs of a patient may be ventilated with the breathable gas that may be accumulated and/or mixed in the accumulator). Moreover, in some embodiments, the flow of the blended breathable gas, which may have a selected FiO2, into the lungs of the patient may be controlled to produce a selected pressure and/or to provide a selected amount flow (e.g., mass per unit time) into the lungs of the patient.

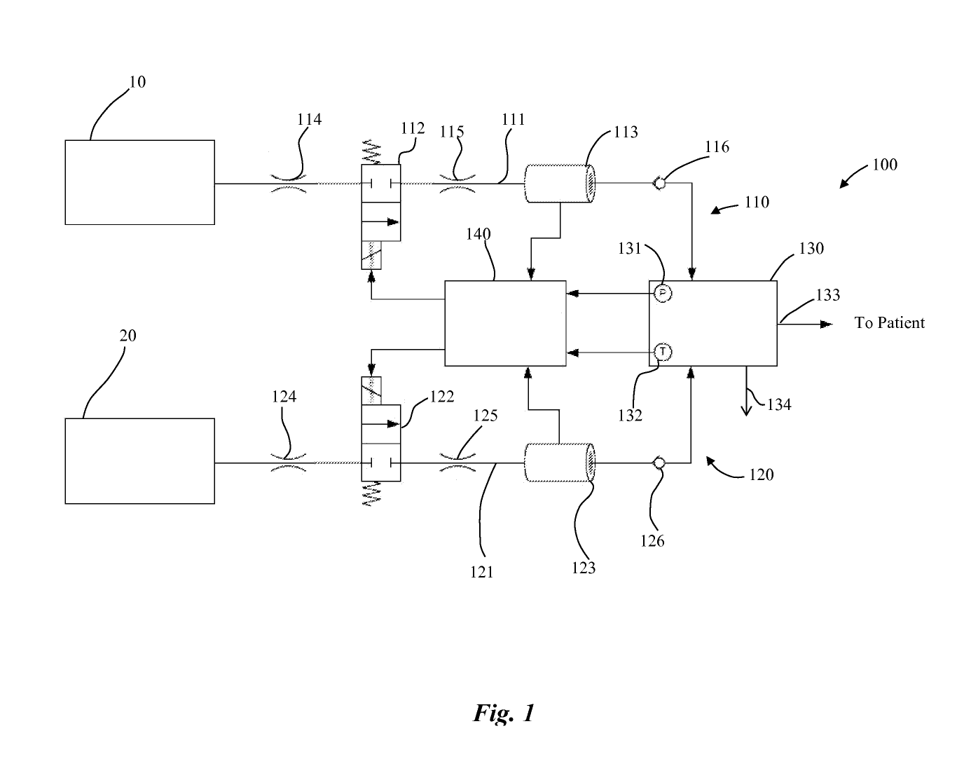

FIG. 1 is a schematic illustration of a fluid blending system 100 according to an embodiment. Specifically, the fluid blending system 100 may supply mixed or blended breathable gas to the lungs of the patient (e.g., the breathable gas supplied by the fluid blending system 100 may have a selected FiO2). The fluid blending system 100 may couple to or include a first fluid supply 10 and a second fluid supply 20 to receive the respective first and second fluids therefrom for mixing and producing a blended fluid, such as breathable gas having a selected FiO2. From the first fluid supply 10 and second fluid supply 20, the first and second fluids may flow into a first fluid supply branch 110 and into a second fluid supply branch 120, respectively.

Generally, the first fluid supply branch 110 may include one or more supply lines 111 that may couple the first fluid supply 10 in fluid communication with the mixing location, and the second fluid supply branch 120 may include one or more supply lines 121 that may couple the second fluid supply 20 in fluid communication with the mixing location. The supply lines 111 and/or the supply lines 121 may include or couple multiple elements and/or components of the fluid blending system 100 (e.g., the elements and/or components connected by the supply lines 111 and 121 may regulate, sense, or otherwise interact with the flow of fluid in the respective first and second fluid supply branches 110 and 120, as described below in more detail. It should be appreciated that the supply lines 111 and/or the supply lines 121 may have any suitable size (e.g., cross-sectional size) and/or shape, which may vary from one embodiment to the next.

In some embodiments, the first fluid supply branch 110 and the second fluid supply branch 120 may be similar or the same as each other. For example, the first fluid supply branch 110 and the second fluid supply branch 120 may have the same or similar elements and/or components, which may be arranged in the same or in a similar manner. Moreover, in some embodiments, the lengths of the first fluid supply branch 110 and second fluid supply branch 120 may be similar or the same (e.g., the supply lines 111 and 121 of the first and second fluid supply branches 110 and 120 may have similar or the same lengths).

Generally, the first and second fluids in the first fluid supply branch 110 and the second fluid supply branch 120 may flow to a mixing location where a mixed or blended fluid, such as the blended breathable gas of a selected FiO2, may be produced. In the illustrated embodiment, the first fluid supply branch 110 and the second fluid supply branch 120 are in fluid communication with an accumulator 130, and the first and second fluids flow from the first fluid supply branch 110 and the second fluid supply branch 120, respectively, into the accumulator 130 (e.g., the accumulator 130 defines or includes an accumulator volume that is in fluid communication with the supply lines 111 and supply lines 121 of the respective first fluid supply branch 110 and second fluid supply branch 120). The fluid blending system 100 may include various elements or components that may regulate fluid flow in the first fluid supply branch 110 and/or in the second fluid supply branch 120, such that selected amounts (e.g., masses of fluids, volumes of fluids, masses of fluids per unit time, volumes of fluids per unit time, etc.) of the first and second fluids enter the accumulator 130 and mix in the accumulator 130 to produce the blended fluid, such as the breathable gas, which has a selected composition.

In particular, in at least one embodiment, the first fluid supply branch 110 of the fluid blending system 100 may include a first branch valve 112, and the first fluid supply branch 110 may include a second branch valve 122, which may control the flow of the first and second fluids from the first fluid supply 10 and second fluid supply 20 into the respective first fluid supply branch 110 and second fluid supply branch 120 (e.g., the first branch valve 112 and/or the second branch valve 122 may be electrically-controlled valves, such as solenoid valves). For example, the fluid blending system 100 may include a controller 140 that may operate and/or direct operation of the first branch valve 112 and second branch valve 122. Accordingly, for example, the controller 140 may control the amount of the first fluid passing through the first branch valve 112 and/or the amount of the second fluid passing through the second branch valve 122 and into the accumulator 130.

Generally, the controller 140 may include a processor, and I/O interface, and a memory coupled to the processor and including instructions stored thereon for performing acts or steps for operating component(s) of the fluid blending system 100 according to one or more of the embodiments described herein. Additionally or alternatively, the controller 140 may include field programmable gate arrays (FPGAs) that may be configured or programmed to perform one or more acts or steps for operating component(s) of the fluid blending system 100 according to one or more embodiments described herein. For example, one or more of the sensors described herein may be operably coupled to the controller 140 at the I/O interface thereof, such that the controller 140 may receive signal from the sensor(s). The controller 140 may process the received signals according to one or more embodiments (e.g., the controller 140 may include A/D converter and may convert analog signals to one or more digital signals, such that the processor may perform one or more acts or steps according to one or more embodiments described herein).

In some embodiments, the controller 140 may fully open (directly or indirectly) the first branch valve 112 and/or the second branch valve 122 for a determined or selected amount of time, such that a correspondingly determined or selected amount of fluid passes through the first branch valve 112 and/or second branch valve 122. For example, the first and second branch valves 112, 122 may be periodically opened and closed to allow a selected amount of fluid to pass therethrough. Additionally or alternatively, the first branch valve 112 and/or second branch valve 122 may be proportional valves, and the controller 140 may partially open the first branch valve 112 and/or the second branch valve 122 to produce a fluid flow therethrough of a selected amount and/or mass. For example, the controller 140 may receive an input related to a selected composition for a blended fluid (e.g., the input may be related to the FiO2 of a breathable gas). Based at least partially on the received input, the controller 140 may operate or direct operation of the first branch valve 112 and second branch valve 122 to produce a flow of the first and second fluids of selected or suitable amounts in the first fluid supply branch 110 and the second fluid supply branch 120 and into the accumulator 130, such as to produce the blended fluid of the selected composition.

In an embodiment, the first branch valve 112 and/or the second branch valve 122 may be operated at least partially based on a formula or a lookup table. For example, the controller 140 may correlate opening a closed valve (e.g., the first branch valve 112 and/or the second branch valve 122) for a specified amount of time to allow a corresponding amount of fluid to flow therethrough during the operation. In some embodiments, to open and close the first branch valve 112 and/or the second branch valve 122, the controller 140 may reference a lookup table that may list valve opening times and correspondence to fluid pressure at and/or temperature the inlet of the valve and an amount of fluid to pass through the valve. Alternatively or additionally, the controller 140 may use a formula to determine the opening and closing times for the valves, which may be based on the fluid pressure and/or temperature at the inlet of the valve and the amount of fluid to pass through the valve.

In at least one embodiment, the first branch valve 112 and/or the second branch valve 122 may be proportional valves and may be opened to define any selected opening therethrough (e.g., the first branch valve 112 and/or second branch valve 122 may partially open in a manner that is proportional to the signal applied thereto). For example, to operate or direct operation of the first branch valve 112 and/or the second branch valve 122, the controller 140 may reference a lookup table that may list the valve openings (e.g., by percentage and/or by the amount of signal required, such as voltage and/or amperage) correlated with the fluid pressure and/or temperature at the inlet of the valve and the amount of fluid to pass through the valve. Alternatively or additionally, the controller 140 may use a formula to determine the openings (e.g., by percentage and/or by the amount of signal required, such as voltage and/or amperage) for the valves, which may be based on the fluid pressure and/or temperature at the inlet of the valve and the amount of fluid to pass through the valve.

In some embodiments, the fluid blending system 100 may include one or more sensors that may be operably coupled to the controller 140. For example, the controller 140 may receive signals from the one or more sensors, which may be related to pressure of the fluid, temperature of the fluid, amount of flow of the fluid, or combinations thereof (e.g., the sensors may be in fluid communication with the first fluid supply branch 110, second fluid supply branch 120, accumulator 130, or combinations thereof). For example, the fluid blending system 100 may include pressure and/or temperature sensor(s) at or near the inlet of the first branch valve 112 and/or near the inlet of the second branch valve 122. Additionally or alternatively, one or more pressure and/or temperature sensors may be positioned at or near the first fluid supply 10 and/or second fluid supply 20. In any event, the controller 140 may receive signals from the pressure and/or temperature sensors and may operate or direct operation of the first branch valve 112 and/or second branch valve 122 (e.g., in a manner described above).

In some embodiments, the fluid blending system 100 may include one or more mass flow sensors, which may detect the amount of fluid flow in the first fluid supply branch 110 and/or second fluid supply branch 120 (e.g., the mass flow sensor(s) may sense or detect the mass of the fluid flowing therethrough). In the illustrated embodiment, the fluid blending system 100 includes mass flow sensors 113 and 123 positioned on the first fluid supply branch 110 and second fluid supply branch 120, respectively. For example, each of the mass flow sensors 113 and 123 may include at least one of a hot-wire mass flow sensor, a hot MEMS mass flow sensor, or an ultrasonic mass flow sensor. As the controller 140 receives signals from the respective mass flow sensors 113 and 123, the controller 140 may determine the amounts (or an estimated amounts) of first and second fluids that enter the accumulator 130 (e.g., by integrating over time the amounts of detected fluids based on the signals received from the mass flow sensors 113 and 123). Accordingly, the controller 140 may operate and/or direct operation of the first branch valve 112 and second branch valve 122 to produce a suitable or selected flow of the first and second fluids in the corresponding first and second fluid supply branches 110, 120, such as to produce a suitable or selected concentration ratio of the first and second fluids in the blended fluid inside the accumulator 130.

As described above, the controller 140 may receive input (e.g., the controller 140 may receive input from a user) that may be related to a selected target concentrations of the first and second fluids in the blended fluid (e.g., the FiO2 of the breathable gas). Accordingly, the controller 140 may operate the first branch valve 112 and/or the second branch valve 122 in a manner that produces the flow of the first and second fluids in the respective first fluid supply branch 110 and second fluid supply branch 120 suitable or selected to produce the selected composition of the blended fluid in the accumulator 130. In particular, for example, the controller 140 receives signals from the mass flow sensors 113 and 123 and may determine the amounts of the first and second fluids entering the accumulator 130. At least partially based on the amount of the first and second fluids determined to be entering the accumulator 130, the controller 140 may operate or direct operation of the first fluid supply branch valve 112 and/or second fluid supply branch valve 122 in a manner that modifies the flow the first and second fluids to produce the selected composition of the blended fluid in the accumulator 130. For example, the controller 140 may substantially continuously or intermittently compare the determined concentration in the accumulator 130 (e.g., based on the masses of the first and second fluids detected by the mass flow sensors 113 and 123) to the selected target concentration for the blended fluid and may operate the first branch valve 112 and the second branch valve 122 in a manner that produces suitable flow in the first and second fluid supply branches 110 and 120 to produce the target concentration.

In some embodiments, the controller 140 may directly or indirectly open and close the first and/or second branch valves 112, 122 (e.g., fully open and fully close) for selected periods of time to produce the flow of the first and second fluids in the respective first fluid supply branch 110 and second fluid supply branch 120 in a manner that produces the blended fluid in the accumulator 130, which has the target concentration of the first and second fluids. Alternatively or additionally, the controller 140 may operate or direct operation of the first branch valve 112 and/or second branch valve 122 to configure the first branch valve 112 and/or second branch valve 122 to be partially open, partially close, fully open, fully close, or combinations of the foregoing to produce suitable flow of the first and second fluids and consequently to produce the target concentration or composition of the blended fluid in the accumulator 130. It should be appreciated that first branch valve 112 and/or the second branch valve 122 may be partially opened and/or partially closed define opening of a suitable size produce the suitable flow of the first and second fluids and respective first fluid supply branch 110 second fluid supply branch 120.

In some embodiments, the fluid blending system 100 may include pressure and/or temperature sensors in fluid communication with the internal volume of the accumulator. For example, the fluid blending system 100 may include a pressure sensor 131 and a temperature sensor 132 positioned inside the accumulator 130. The pressure sensor 131 and/or the temperature sensor 132 may be operably coupled to the controller 140, such that the controller 140 receives signals therefrom. In an embodiment, the controller 140 at least partially closes or directs closing of the first branch valve 112 and/or second branch valve 122 when the pressure and/or temperature and the volume of the accumulator 130 nears or reaches a threshold value (e.g., as detected by the pressure and temperature sensors 131 and/or 132). For example, the controller 140 may close or direct closing of the first branch valve 112 and/or second branch valve 122 to prevent the pressure inside the accumulator 130 from reaching dangerously high levels at which the accumulator 130 and/or one or more components downstream therefrom may be damaged or broken. However, it should be noted that in other embodiments, the controller 140 may control the fluid blending system 100 without taking into account the pressure and/or temperature in the accumulator 130.

As mentioned above, the controller 140 may directly or indirectly operate the first branch valve 112 and second branch valve 122 to produce a suitable or selected flow of the first and second fluids, such that mixing thereof would produce a blended fluid having a selected proportion of the first and second fluids (e.g., a blended breathable having a selected FiO2). Under some operating conditions, the blended fluid may be periodically or continuously leaving the accumulator 130, such as flowing out of an output outlet 133 (e.g., as may be controlled by an output valve) and to the lungs of the patient. For example, the output outlet 133 may be coupled to a ventilator mask that is worn by the patient. As described below in more detail, for example, the controller 140 and/or another controller may control operation of the output valve to control the flow of the blended fluid out of the accumulator 130.

Moreover, under some operating conditions, the blended fluid may require change in proportions of the first and second fluids. For example, a patient receiving a blended breathable gas from the accumulator 130 may require a breathable gas of a different FiO2 than contained in the accumulator 130. In at least one embodiment, the controller 140 may operate or direct operation of the first branch valve 112 and/or second branch valve 122 to change the amounts of the first and second fluids flowing in the respective supply lines 111 and 121 from first relative amounts (or proportions) to second relative amounts (or proportions), thereby adjusting the proportions of the first and second fluid entering the accumulator 130. For example, the controller 140 may adjust the proportion of the first and second fluid entering the accumulator 130 to a final proportion or a new concentration ratio of the target or selected blended fluid (e.g., of the breathable gas). As such, for example, as the changed amounts of the first and second fluids enter the accumulator 130, and the blended fluid of a previously selected concentration ratio exits the accumulator 130 and/or mixes with the additional first and second fluids that enter the accumulator 130, the concentration ratio of the blended fluid in the accumulator 130 may gradually change toward the new concentration ratio. Alternatively or additionally, the controller 140 may estimate the amount or mass of the blended fluid in the accumulator 130, which has a previously selected concentration ratio and may determine the amount of the first and second fluids to flow into the accumulator 130 to produce the blended fluid therein that has the new concentration ratio. For example, the fluid flow out of the accumulator 130 may be stopped or the controller 140 may determine the amount of the first and second fluids to flow into the accumulator 130 accounting for the diminishing amount of the blended fluid that has the previously selected concentration ratio, as such fluid leaves the accumulator 130.

Alternatively or additionally, the accumulator 130 may include a purge outlet 134 for evacuating the blended fluid that has a first concentration ratio of the first and second fluids. In some embodiments, a purge valve (not shown) may be opened (e.g., the controller 140 may open or direct opening of the purge valve) and the blended fluid having the first concentration ratio may be evacuated or purged from the accumulator 130 as the first and second fluid flow into the accumulator 130 (e.g., the amounts or masses, such as masses per unit time, of the first and second fluid flowing into the accumulator 130 may be selected to produce a blended fluid of a second concentration ratio, which is different than the first concentration ratio). For example, the purge valve may remain open for an amount of time selected or suitable to change the concentration of the blended fluid (e.g., the amount of time may be determined by the controller 140 as a suitable time for evacuating and replacing the blended fluid of the first concentration with the blended fluid of the second concentration). As another example, the purge valve may remain open for an amount of time selected or suitable to lower the pressure in the accumulator 130 should the pressure sensor 131 indicate that the pressure is too high.

Under some operating conditions, the blended fluid of a first composition may be evacuated from the accumulator 130 and/or replaced by a blended fluid of a second composition. For example, the composition may be changed to include 100% of the first or second fluids (e.g., the FiO2 may be changed from 20% oxygen to 100% oxygen). In an embodiment, the controller 140 may open or direct opening of the purge valve as the modified amounts of the first and second fluids flow to the accumulator 130 (e.g., 100% of the first fluid or 100% of the second fluid).

In an embodiment, the fluid blending system 100 may include one or more orifices positioned on the first fluid supply branch 110 and/or the second fluid supply branch 120. For example, the fluid blending system 100 may include an orifice 114 downstream from the first fluid supply 10 on the first fluid supply branch 110 (e.g., upstream from the first branch valve 112). Additionally or alternatively, the fluid blending system 100 may include an orifice 124 downstream from the second fluid supply 20 on the second fluid supply branch 120 (e.g., upstream from the second branch valve 122). Under some operating conditions, the orifice 114 and/or the orifice 124 may protect the respective first branch valve 112 and second branch valve 122 from high pressures and/or may normalize or reduce pressure of the respective first and second fluids exiting the first fluid supply 10 and second fluid supply 20. However, it should be noted that the orifices 114 and 124 may be omitted in some embodiments.

Additionally or alternatively, the fluid blending system 100 may include one or more orifices downstream from the first branch valve 112 and/or second branch valve 122. For example, the fluid blending system 100 may include orifices 115 and 125 downstream from the first branch valve 112 and second branch valve 122 along the first fluid supply branch 110 and second fluid supply branch 120, respectively. More specifically, the orifices 115 and 125 may further reduce the pressure of the first and second fluids downstream from the first branch valve 112 and second branch valve 122.

In some embodiments, the fluid blending system 100 may include one or more check valves that may limit or prevent upstream or return flow of the first and/or the second fluid from the accumulator 130. For example, the fluid blending system 100 may include a one-way check valve 116 positioned along the first fluid supply branch 110 (e.g., near the accumulator 130). The fluid blending system 100 may also include a one-way check valve 126 positioned along the second fluid supply branch 120 (e.g., near the accumulator 130).

It should be noted that in some embodiments, various components of the fluid blending system 100 may be omitted. For example, at least of, two or more, or all of the mass flow sensors 113 and 123, the orifices 115 and 125, or one-way check valves 116 and 126 may be omitted in some embodiments. As another example, the mass flow sensors 113 and 123, the orifices 115 and 125, and one-way check valves 116 and 126 may be omitted such that respective fluids may flow from the first branch valve 112 and the second branch valve 122 directly to the accumulator 130.

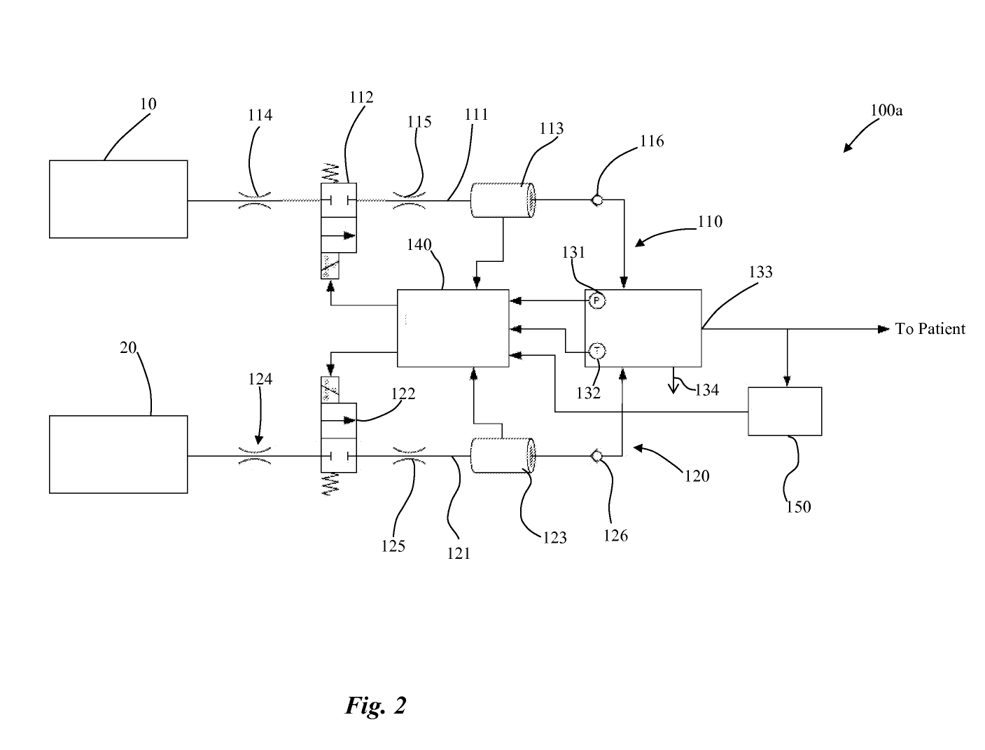

In some embodiments, the fluid blending system may include a fluid concentration sensor in fluid communication with the blended fluid in the accumulator. FIG. 2 is a schematic diagram of a fluid blending system 100a that includes the accumulator 130 and a fluid concentration sensor 150 in fluid communication with the blended fluid in the accumulator 130, according to an embodiment. Except as described herein, the fluid blending system 100a and its elements and components may be similar to or the same as the fluid blending system 100 (FIG. 1) and its corresponding elements and components.

For example, the fluid concentration sensor 150 may be positioned at an output outlet 133, inside the accumulator 130, at a purge outlet 134, or combinations of the forgoing (e.g., the fluid blending system 100a may include multiple fluid concentration sensors). The fluid concentration sensor 150 may be operably coupled to the controller 140, such that the controller 140 receives signals therefrom, which are related to the concentration ratio of the first and second fluids in the blended fluid inside the accumulator 130. Hence, in some embodiments, the controller 140 may operate or direct operation of the first branch valve 112, second branch valve 122, purge valve, output valve, or combinations thereof based at least partially on the detected concentration of the blended fluid in the accumulator 130.

In some embodiments, the fluid blending system 100 may mix or blend the first and second fluids based on the readings or measurements from the fluid concentration sensor 150, without relying on the readings from the mass flow sensors 113 and/or 123 (e.g., the fluid blending system 100 may be configured without the mass flow sensors 113 and/or the 123). The controller 140 may operate the first branch valve 112 and/or the second branch valve 122 based on a list of correlated values and/or on a formula, as described above. For example, the controller 140 may operate the first branch valve 112 and/or the second branch valve 122, as described above, until the concentration ratio of the blended fluid in the accumulator 130 reaches the selected concentration ratio (e.g., until the concentration ratio of the blended fluid in the accumulator 130 changes from a first concentration ratio to a second concentration ratio). For example, the controller 140 may intermittently fully open and fully close the first branch valve 112 and/or second branch valve 122 for predetermined amounts of time to allow a selected amount of first and second fluids to flow to the accumulator 130, where the amounts are determined or selected by the controller 140 to produce a selected composition of the blended fluid. Additionally or alternatively, the controller 140 may partially open and may vary the size of the opening of the first branch valve 112 and/or second branch valve 122 to allow a selected amount of first and second fluids to flow to the accumulator 130, where the amounts are determined or selected by the controller 140 to produce a selected composition of the blended fluid.

In some embodiments, the fluid concentration sensor 150 may send signal(s) to the controller 140. The signal(s) may be related to the detected concentration of the first and/or second fluids in the blended fluid (e.g., to the ratio of the first and second fluids), and the controller 140 may adjust the openings of the first branch valve 112 and/or the second branch valve 122 to produce a selected composition of the blended fluid. For example, the controller 140 may adjust or direct adjustment of the size of the openings, the frequency of opening and closing the valves, the duration the valve(s) stay open, combinations thereof, etc., to adjust the composition of the blended fluid based at least partially on the signals received from the fluid concentration sensor 150.

In an embodiment, based at least partially on the signal(s) from the concentration sensor 150, the controller 140 may determine that to achieve a selected concentration ratio, the blended fluid requires additional amount of the first fluid. Accordingly, the controller 140 may operate or direct operation of the first branch valve 112 and/or second branch valve 122 to produce an increase in the amount of flow (e.g., mass) of the first fluid relative to the second fluid flowing toward the accumulator 130. Conversely, at least partially based on the signal(s) from the fluid concentration sensor 150, the controller 140 may determine that to achieve a selected concentration ratio, the blended fluid requires additional amount of the second fluid. As such, the controller 140 may operate or direct operation of the first branch valve 112 and/or second branch valve 122 to produce an increase in the amount flow (e.g., mass) of the second fluid relative to the first fluid flowing toward the accumulator 130.

In some embodiments, the controller 140 may maintain the purge valve open while changing the composition of the blended fluid in the accumulator 130. More specifically, as described in an example above, the controller 140 may maintain the purge valve open until the concentration ratio of the blended fluid in the accumulator 130 reaches the selected concentration ratio (e.g., until the concentration ratio of the blended fluid in the accumulator 130 changes from a first concentration ratio to a second concentration ratio). Moreover, the controller 140 may close or direct closing of the purge valve when the selected concentration ratio is detected in the blended fluid inside the accumulator 130.

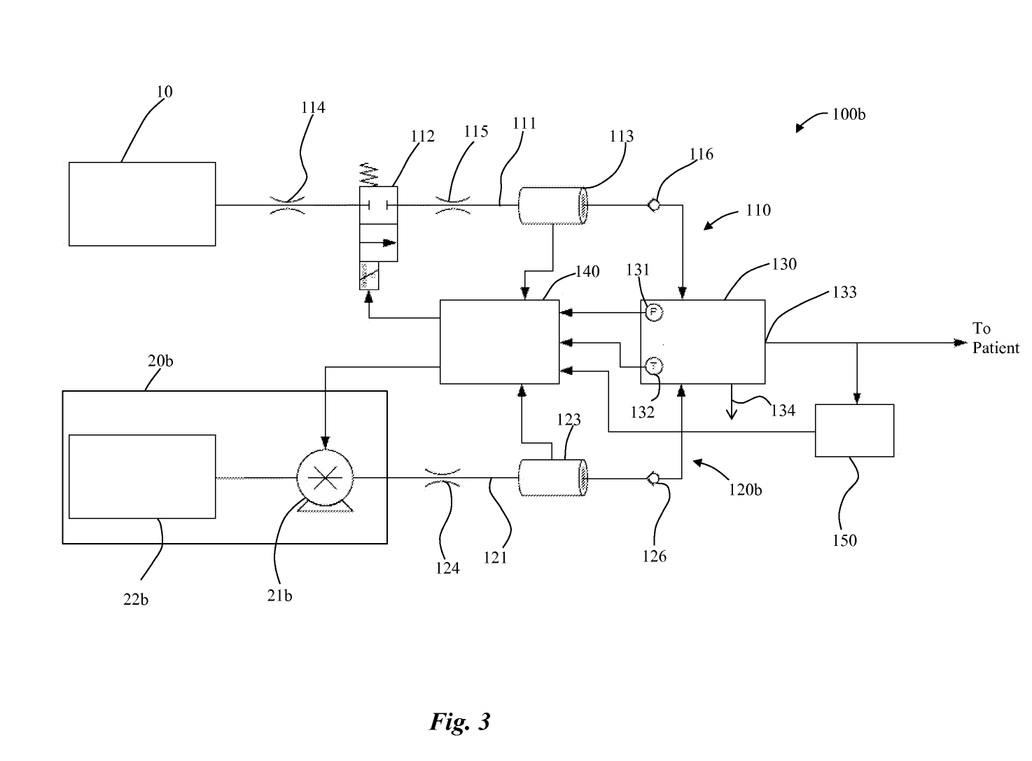

As described above, the first fluid supply 10 and/or second fluid supply 20 may supply pressurized fluids into the first fluid supply branch 110 and second fluid supply branch 120, respectively. In some embodiments, the first or second fluid supplies may include a compressor or a pump coupled to a source of unpressurized fluid, and the pump may pressurize the fluid supplied into one or more of the branches of the fluid blending system. FIG. 3 is a schematic illustration of a fluid blending system 100b that includes or couples to a second fluid supply 20b that includes a compressor 21b coupled to a source of unpressurized second fluid 22b, according to an embodiment. Except as described herein, the fluid blending system 100b and its elements and components may be similar to or the same as any of the fluid blending system 100, 100a (FIGS. 1, 2) and their corresponding elements and components.

In some embodiments, the controller 140 may be operably coupled to the compressor 21b and may control operation thereof. For example, the controller 140 may cycle the compressor 21b between on and off states. Moreover, in at least one embodiment, the controller 140 may control the output pressure and/or the amount of fluid flowing from the compressor 21b. For example, the controller 140 may control the RPM of a motor of the compressor 21b, thereby controlling the output from the compressor 21b into a second fluid supply branch 120b (e.g., controlling the pressure and the amount of fluid supplied by the compressor 21b).

Generally, the second fluid supply branch 120b may be similar to or the same as the second fluid supply branch 120 (FIG. 1). For example, the second fluid supply branch 120b may include orifices 124 and/or 126 located thereon (e.g., downstream from the second fluid supply 20b). In some embodiments, the second fluid supply branch 120b may include the orifice 124 located downstream from the second fluid supply 20b. In at least one embodiment, the second fluid supply branch 120b may be configured without a branch valve for controlling the flow of the second fluid from the second fluid supply 20b in the second fluid supply branch 120b. For example, the controller 140 may control the compressor 21b to produce a suitable amount or mass of the flow, suitable pressure, etc., in the second fluid supply branch 120b.

Moreover, the controller 140 may control operation of the compressor 21b (e.g., the amount of pressure and/or mass of the fluid supplied by the compressor 21b into the second fluid supply branch 120b) in the same or similar manner as described above in connection with the branch valves. For example, the controller 140 may receive signals from the mass flow sensor 123 that may relate to the amount or mass of the second fluid passing at the location of the mass flow sensor 123 in the second fluid supply branch 120b. The controller 140 may (directly or indirectly) operate the compressor 21b to produce a suitable or selected amount of the flow (e.g., mass) of the second fluid, which may be detected at the location of the mass flow sensor 123.

As described above, in some embodiments, the first fluid supply branch 110 and the second fluid supply branch 120b may be similar or the same to each other. For example, the first fluid supply branch 110 of the fluid blending system 100b may be similar to or the same as the first fluid supply branch 110 of the fluid blending system 100 (FIG. 1). Alternatively, however, the first fluid supply branch 110 of the fluid blending system 100b may be the same as the second fluid supply branch 120b. For example, the first fluid supply branch 110 may include or may be coupled to a compressor or pump that may be coupled to a supply of unpressurized fluid. Moreover, the controller 140 may control operation of the compressor or pump, thereby controlling the pressure and/or the amount of the flow of the first fluid supplied into the first fluid supply branch 110 by the compressor or pump (as may vary from one embodiment to the next).

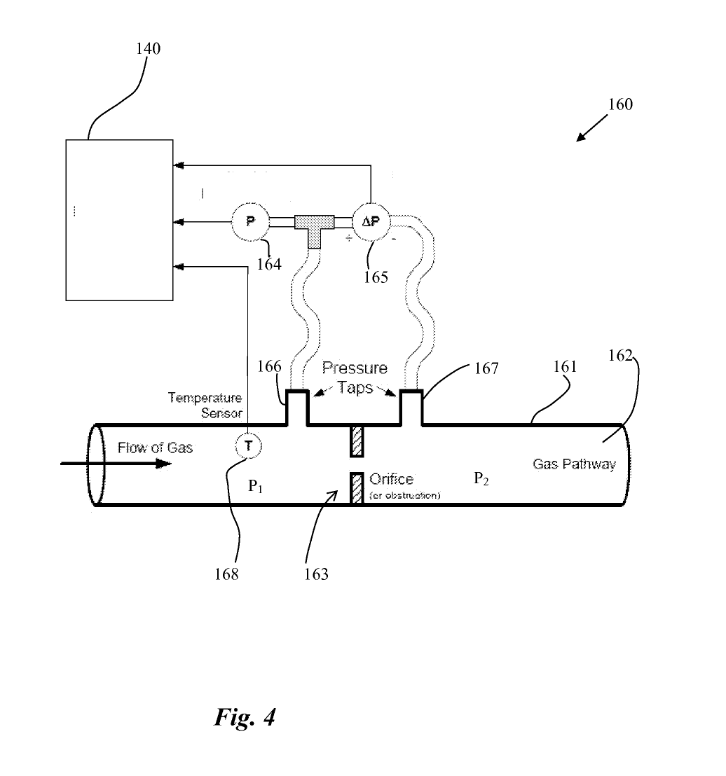

Generally, the fluid blending system may include any number of suitable mass flow sensors, which may vary from one embodiment to the next. Moreover, the mass flow sensors may be coupled to or integrated with one or more of the supply lines of the fluid blending system and/or with one or more other elements or components (e.g., one or more orifice of the blending system may be included in or may form a portion of the mass flow sensor). FIG. 4 is a schematic illustration of a mass flow sensor 160 according to an embodiment, which may be used for any of the mass flow sensors in any of the embodiments disclosed herein. The mass flow sensor 160 may be configured to produce one or more signals related to the mass of the fluid flowing therethrough, which may be processed by the controller 140 to determine the amount of flow of the fluid per unit of time and/or the total amount of fluid that entered the accumulator 130 during a selected time period. Except as otherwise described herein, the mass flow sensor 160 and its elements and components may be similar to or the same as any of the mass flow sensors 113, 123 (FIGS. 1-3) and their corresponding elements and components.

In an embodiment, the mass flow sensor 160 may include an enclosure 161 that defines a lumen 162 for a fluid flow therethrough. As mentioned above, the enclosure 161 may be integrated with and/or may form part of a fluid supply line in the fluid blending system. The fluid in the lumen 162 may flow therein as indicated with the arrow (shown in FIG. 4) and may pass through an orifice 163 that may obstruct fluid flow in the lumen 162 and/or reduce the pressure of the fluid from a first pressure P₁ to a second pressure P₂ (e.g., the fluid upstream from the orifice 163 may be at the first pressure P₁, and the fluid downstream from the orifice 163 may be at the second pressure P₂).

The mass flow sensor 160 may include one or more sensors for determining the first and second pressures P₁, P₂. In particular, one or more pressure sensors may be in fluid communication with an upstream portion of the lumen 162 that is upstream from the orifice 163 (e.g., in fluid communication with the portion of the fluid that has the first pressure P₁), and one or more pressure sensors may be in fluid communication with a downstream portion of the lumen 162 that is downstream from the orifice 163 (e.g., in fluid communication with the portion of the fluid that has the second pressure P₂). In an embodiment, the mass flow sensor 160 may include a first pressure sensor 164 in fluid communication with and configured to sense or detect the pressure of the fluid that is at the first pressure P₁, and a second pressure sensor 165 in fluid communication with and configured to sense or detect the pressure of the fluid that is at the first pressure P₂. For example, the first pressure sensor 164 and the second pressure sensor 165 may be coupled to the enclosure 161 at respective first and second ports 166, 167 located on opposing sides of the orifice 163, such that the first pressure sensor 164 is in fluid communication with the fluid located upstream from the orifice 163 and the second pressure sensor 165 is in fluid communication with the fluid downstream from the orifice 163. In an embodiment, the first and second pressure sensors 164 and 165 may send signals to the controller 140, which may be related to the first and second pressures P₁ and P₂ sensed thereby.

Generally, the specific pressure sensors may vary from one embodiment to the next. In some embodiments, the pressure sensors may be piezoelectric, piezoresistive, capacitive, MEMS, LVDT, combinations thereof, etc. Moreover, the pressure sensors may send digital or analog signals to the controller 140.

In at least one embodiment, the mass flow sensor 160 may include at least one temperature sensor that may determine the temperature of the fluid in the lumen 162. For example, the mass flow sensor 160 may include a temperature sensor 168 that may be positioned and configured to detect the temperature in the fluid (e.g., the temperature sensor 168 may be positioned in the lumen 162, such as downstream from the orifice 163). The temperature sensor 168 may be coupled to the controller 140 and may send signals thereto, which may be related to the temperature sensed thereby (e.g., to a first temperature T₁ of the first fluid located upstream from the orifice 163).

The specific temperature sensor(s) may vary from one embodiment to the next. For example, the temperature sensor may include at least one of a thermocouple, a thermistor, a resistance temperature detector (RTD), or a silicon temperature sensor positioned in contact with the fluid in the lumen 162. In any event, the temperature sensor may send signal(s) to the controller 140, which may be related to the temperature of the fluid at the location of the sensor.

In an embodiment, the controller 140 may determine or estimate amount of the fluid flow through the orifice 163 (e.g., mass of fluid per unit of time). For example, the orifice 163 may be sized and configured to permit a determined or selected amount of fluid therethrough depending on the pressure, density, etc., of the fluid. Specifically, the amount of flow through the orifice 163 may be generally constant for a specific pressure and/or type of fluid (e.g., fluid characteristics, such as density, etc.) flowing through the orifice 163. Accordingly, for example, the controller 140 may determine the amount of flow of the fluid in the lumen 162 based on the first pressure P₁. In an embodiment, the flow amounts for different pressures and/or different fluids may be determined empirically to calibrate the controller 140.

Moreover, the controller 140 may determine the mass per unit time of the fluid flow in the lumen 162. For example, the characteristics of the fluid in the lumen 162 may be approximated based on the Ideal gas law. In at least one embodiment, the controller 140 may determine the mass of the fluid flowing in the lumen 162 per unit time based at least partially on the first and/or second pressure P₁, P₂ (e.g., at least partially based on the change in pressure after the fluid passes the orifice 163), on the temperature T₁, or combinations of the foregoing.

In some embodiments, the mass flow sensor 160 may include a second temperature sensor that may be positioned downstream from the orifice 163 (e.g., to determine T₂). For example, the controller 140 may determine the mass of flowing fluid per unit time in the lumen 162 based at least partially on the first and/or second pressures P₁, P₂ (e.g., the change in pressure after the fluid passes the orifice 163), on the first and/or second temperatures T₁, T₂, or combinations of the foregoing.

Moreover, the controller 140 may determine the mass of flowing fluid per unit time based on a formula or a system of formulas that the controller 140 may continuously or substantially continuously process to determine the mass of fluid flowing per unit time. Additionally or alternatively, the controller 140 may include a lookup table that may have the values for the mass of flowing fluid per unit time corresponding to one or more combinations of the first and/or second pressures P₁, P₂ (e.g., the change in pressure after the fluid passes the orifice 163), on the first and/or second temperatures T₁, T₂, or combinations thereof.

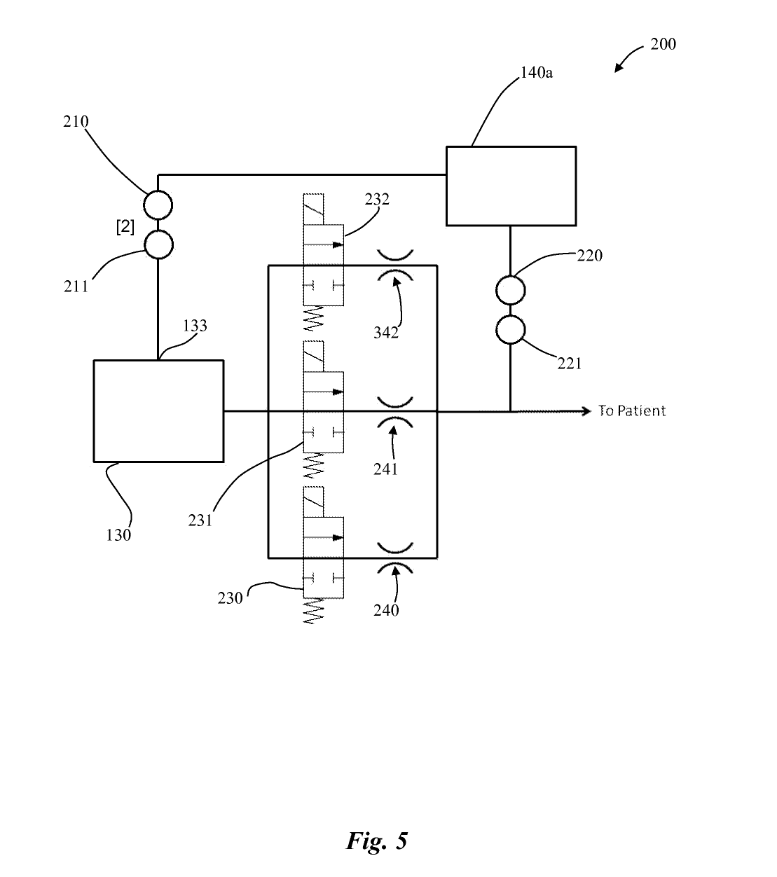

As described above, the fluid entering the accumulator may be breathable gas. Moreover, the breathable gas may be supplied from the accumulator to the lungs of patient, such as via a ventilator mask. FIG. 5 is a schematic illustration of an embodiment of a flow control circuit 200 that may facilitate supplying the breathable gas from the accumulator 130 into the lungs of the patient. It should be appreciated, that the flow control circuit 200 may be coupled to any fluid blending system, including any of the fluid blending systems disclosed herein.

Generally, the accumulator 130 may be filled with a breathable gas of a selected composition (e.g., of a selected FiO2) and to any suitable or selected pressure (e.g., from 5 psi to 20 psi), which may vary from one embodiment to the next. Moreover, the accumulator 130 may be substantially continuously filled with the first and/or second fluids (e.g., with atmospheric air and oxygen) or may be filled between breaths, such that the flow into the accumulator 130 is paused when the breathable air is supplied into the lungs of the patient and resumes when the patient exhales and/or the exhaled gas is removed from the lungs of the patient. It should be also appreciated that multiple breaths may be taken by or provided to the patient before additional breathable gas is added into the accumulator 130.

Under some operating conditions, the supply of breathable gas in the accumulator 130 may at least periodically diminish, as the breathable gas exits the accumulator 130 and flows into the lungs of the patient. In an embodiment, the flow control circuit 200 may regulate flow of the breathable gas out of the accumulator 130 in a manner that the pressure and/or the amount of breathable gas (e.g., the mass and/or the volume of the breathable gas per unit time) provided to the lungs of the patient may be at a selected level, such as a selected fixed or constant level. For example, the flow control circuit 200 may include pressure and flow sensors 210, 211 positioned in the accumulator 130 or near the output outlet 133 of the accumulator 130, such as to detect or determine the pressure of the breathable gas inside the accumulator 130 and/or the amount (e.g., volume or mass per unit time) of the breathable gas exiting the accumulator 130. The flow sensors 210 and/or 211 may be operably coupled to a controller 140a. Except as otherwise described herein, the controller 140a may be similar to or the same as the controller 140 (FIGS. 1-3). Moreover, the controller 140 (FIGS. 1-3) may be configured to perform the acts or steps described herein in connection with the controller 140a (e.g., the controller 140a and the controller 140 may be integrated together as a single controller or may be separate controllers that, in some embodiments, may be operably coupled together, or may operate independently of each other).

In an embodiment, the flow control circuit 200 includes patient-side pressure and flow sensors 220, 221, which may determine the pressure and the amount (e.g., volume or mass per unit time) of the breathable gas being supplied into the lungs of the patient. The flow sensors 220 and/or the 221 may be operably coupled to the controller 140a. In some embodiments, the controller 140a may receive signals from the flow sensors 210, 211, 220, 221 and may adjust the flow out of the accumulator 130 in a manner that produces suitable or selected flow into the lungs of the patient (e.g., amount of flow and/or pressure).

In at least one embodiment, the flow control circuit 200 may include multiple orifices and corresponding flow control valves positioned downstream from the accumulator 130 and arranged in a manner that opening a flow control valve produces flow through the orifice that corresponds to that valve. For example, one or more flow control valves may be opened to produce flow of the breathable fluid out of the accumulator 130, through the flow control valves, and subsequently through the orifices that correspond to and are located downstream from the open flow control valves. The flow control valves may be selectively opened to produce a suitable or selected flow through any number of suitable combinations of orifices, thereby producing selected pressure and flow amount (e.g., volume or mass of the breathable gas per unit time) downstream from the orifices and into the lungs of the patient). Moreover, it should be appreciated that the orifices corresponding to the flow control valves may have any number of suitable sizes and/or shapes, which may vary from one orifice to another. Also, combinations and number of orifices may vary from one embodiment to the next.

In the illustrated embodiment, the flow control circuit 200 includes three flow control valves 230, 231, 232 and corresponding or associated orifices 240, 241, 242 located downstream therefrom, such that opening the valve 230 produces flow through orifice 240, opening the valve 231 produces flow through orifice 241, and opening the valve 232 produces flow through orifice 242. One, some, or each of the valves 230, 231, 232 may be operably coupled to the controller 140a, which may control operation thereof. In some embodiments, one, some, or each of the valves 230, 231, 232 may be dual position valves, which may have only open and closed configurations. Alternatively, one, some, or each of the valves 230, 231, 232 may be a proportional valve, which may be partially opened (e.g., in a manner that is proportional to the signal applied thereto). The controller 140a may operate or direct operation of the one, some, or each of the valves 230, 231, 232, such as to produce flow of the breathable gas through corresponding orifices 240, 241, 242.

For example, the controller 140a may direct opening of the valve 230 to produce a selected pressure drop and/or flow of the breathable gas to the patient lungs that corresponds to the pressure drop and/or flow produced when the breathable fluid passes through the orifice 240. The controller 140a may also direct opening of the valves 231 and/or 232 to produce a selected pressure drop and/or flow of the breathable gas to the patient lungs that corresponds to the pressure drop and/or flow produced when the breathable fluid passes through the orifice 241 and/or 242. The controller 140a also may produce a selected flow of the breathable gas into the lungs of the patient by opening two or more of the valves 230, 231, 232, to produce flow of the breathable gas through corresponding ones of the orifices 240, 241, 242 and the corresponding modification of the flow of the breathable gas (e.g., pressure drop and/or volumetric or mass flow change as compared to the flow of the breathable fluid near the accumulator 130 and/or exiting the output outlet 133 of the accumulator 130).

Generally, a user may (directly or indirectly) select the amount of breathable gas, the pressure of the breathable gas, and the concentration (FiO2) of the breathable gas that is supplied into the lungs of the patient. For example, the user may enter or input into the controller (e.g., into the controller 140 (FIGS. 1-3) and/or into the controller 140a (FIG. 5)) values corresponding to the amount of breathable gas, the pressure of the breathable gas, and the concentration (FiO2) of the breathable gas that is supplied into the lungs of the patient, and the controller may regulate the flow of the breathable gas based on such values (e.g., as described above). For example, the user may enter or input values into a user interface of the controller, such as via a touch screen, keypad, or other suitable user interface. Alternatively or additionally, the controller may receive input related to the diagnosis of the patient, the weight of the patient, treatment related parameters, etc., and may correlate such input to the values corresponding to the amount of breathable gas, the pressure of the breathable gas, and the concentration (FiO2) of the breathable gas that is supplied into the lungs of the patient.

For example, the controller may correlate input of a respiratory condition, such as ARDS, COPD, asthma, trauma, etc., and/or other conditions related to the patient, such as body weight, height, etc., to values corresponding to the amount of breathable gas, the pressure of the breathable gas, and the concentration (FiO2) of the breathable gas that is supplied into the lungs of the patient. Moreover, numeric values, such as body weight, height, etc., may be entered as a specific number or as a range. In some embodiments, the controller may include a display or an output. Hence, for example, after user enters conditions into the controller, the display may receive instructions from the controller and may display a list of settings that may correspond to one or more of the following parameters of the breathable gas: the amount (e.g., mass) of flow of the breathable gas, the pressure of the breathable gas, and the concentration (FiO2) of the breathable gas. The controller may receive selection of the displayed parameters (e.g., default settings or parameters corresponding to the entered conditions) and/or may receive one or more entries of the parameters or values not displayed (e.g., user may modify one or more parameters or values).

For example, the default settings or parameters may be based on literature review, studies, etc. For example, the controller may receive an entry of conditions ARDS and patient weight 60 kg, and the controller may correlate and/or determine a volume-targeted control mode, with a tidal volume and respiratory rate based on the ARDSnet protocol and breathable gas containing 100% oxygen. In another example, choosing the conditions as COPD and patient weight 60 kg, a default parameter for a targeted pressure may be an inhale ratio of 1:1.

While various aspects and embodiments have been disclosed herein, other aspects and embodiments are contemplated. The various aspects and embodiments disclosed herein are for purposes of illustration and are not intended to be limiting.

What is claimed is:

1. A system for blending a first fluid and a second fluid to produce a blended fluid, the system comprising:
an accumulator defining an accumulator volume;
a first fluid supply branch including:
a first valve coupleable to a first source of pressurized fluid;
a first supply line coupled to the first input valve and extending downstream therefrom and being in fluid communication with the accumulator volume;
a first mass flow sensor positioned along the first supply and configured to generate a first signal that corresponds to a mass of fluid at a location thereof;
a second fluid supply including:
a second valve coupleable to a second source of pressurized fluid;
a second supply line coupled to the second input valve and extending downstream therefrom and being in fluid communication with the accumulator volume;
a second mass flow sensor positioned along the second supply line and configured to generate a second signal that corresponds to a mass of fluid at a location thereof; and
a controller operably coupled to the first and second mass flow sensors and configured to receive signals therefrom, the controller being configured to operate or direct operation of the first and the second valves, at least partially based on the received signals from the first and second mass flow sensors, to generate a flow of the first and second fluids targeted and produce a blended fluid in the accumulator, the blended fluid having selected concentrations of the first and second fluids.

2. The system of claim 1 wherein at least one of the first valve or the second valve is a proportional solenoid valve that is operably coupled to the controller, and wherein the controller is configured to the operate or direct operation of the at least one of the first or second valve at least partially based on the signals received from one or more of the first mass flow sensor or the second mass flow sensor.

3. The system of claim 2, further comprising one or more of: a pressure sensor positioned and configured to detect a pressure of the blended fluid in the accumulator volume; or a temperature sensor positioned and configured to detect a temperature of the blended fluid in the accumulator volume.

4. The system of claim 3 wherein one or more of the pressure sensor or the temperature sensor are operably coupled to the controller, the controller configured to receive one or more signals from the one or more of the pressure sensor or the temperature sensor, the controller further configured to close one or more of the first valve or the second valve at least partially based on the signal received from the one or more additional sensors.

5. The system of claim 1, further comprising one or more non-return check valves positioned along one or more of the first supply line or the second supply line.

6. The system of claim 1 wherein the accumulator includes a fluid output outlet for supplying the blended gas to the patient and a purge outlet for purging the blended gas out of the accumulator volume.

7. The system of claim 6, further comprising an output valve positioned and configured to regulate flow out of the fluid output outlet and a purge valve positioned and configured to regulate flow out of the purge outlet.

8. The system of claim 6, further comprising two or more control valves downstream from and in fluid communication with the output outlet of the accumulator, the two or more control valves being operably coupled to the controller.

9. The system of claim 8, further comprising two or more orifices each of which is positioned downstream from a corresponding one of the two or more control valves, wherein the controller is configured to selectively operate the two or more control valves to produce flow through corresponding one of the two or more orifices.

10. The system of claim 1 wherein the first and the second supply branches are configured substantially the same.

11. The system of claim 1 wherein at least one of the first or the second mass flow sensor includes an enclosure defining a lumen and an orifice configured to obstruct fluid flow through the lumen, the mass flow sensor further including a pressure sensor positioned and configured to detect a first pressure in the fluid flow upstream from the orifice.

12. The system of claim 11 wherein the at least one of the first or second mass flow sensors includes another pressure sensor positioned and configured to detect a second pressure of the fluid flow downstream from the orifice.

13. The system of claim 12 wherein the controller is configured to determine a mass of fluid at the at least one of the first or second mass flow sensors at least partially based on one or more of the first pressure, the second pressure, or a temperature of the fluid.

14. The system of claim 13 wherein the at least one of the first or second mass flow sensors includes one or more temperature sensors configured to detect temperature of the fluid flow upstream from the orifice or downstream from the orifice.

15. A system for blending a first gas and a second gas to produce a breathable gas having a selected fraction of inspired oxygen (FiO2), the system comprising:
an accumulator defining an accumulator volume;
a first fluid supply branch including:
an electrically-controlled valve coupled to a source of the first gas;
a first supply line coupled to the electrically-controlled valve and extending downstream therefrom and being in fluid communication with the accumulator volume;
a second fluid supply including a second supply line in fluid communication with the accumulator volume; and
a controller operably coupled to the electrically-controlled valve and configured to direct progressive opening and closing thereof.

16. The system of claim 15, further comprising one more compressors coupled to one or more of the source of the first gas or the source of the second gas, an output of the one or more compressors being coupled to one or more of the first supply line or the second supply line.

17. The system of claim 16 wherein the controller is operably coupled to the one or more compressors and configured to control operation thereof in a manner that produces one or more of a selected pressure or an amount of fluid flow therefrom.

18. The system of claim 16 wherein the controller is configured to direct progressive opening and closing of the electrically-controlled valve at least partially based on the signals received from the fluid concentration sensor.

19. The system of claim 16, further comprising:
another electrically-controlled valve coupled to a source of the second gas, wherein the second supply line is coupled to the electrically-controlled valve and extends downstream therefrom; and
wherein the controller is configured to direct progressive opening and closing of the another electrically-controlled valve at least partially based on the signals received from the fluid concentration sensor.

20. The system of claim 15, further comprising:
one or more of a first mass flow sensor positioned along the first supply line and configured to detect to generate one or more first signals that correspond to a mass of fluid at a location thereof; or
a second mass flow sensor positioned along the second supply line and configured to detect to generate one or more second signals that correspond to a mass of fluid at a location thereof;
wherein the controller is configured to direct progressive opening and closing of the electrically-controlled valve at least partially based on the one or more of one or more first signals or one or more second signals.

21. The system of claim 15, further comprising a fluid concentration sensor in fluid communication with the accumulator volume and configured to detect a concentration of one or more of the first gas or the second gas in the blended breathable gas in the accumulator volume, wherein the controller is operably coupled to the fluid concentration sensor and configured to receive signals therefrom.
